(19) United States
(12) Patent Application Publication
(10) Pub. No.: US 2016/0324614 A1
(43) Pub. Date: Nov. 10, 2016

(54) Animal Protection Device

(71) Applicant: Su-Ling Cheng, New Taipei City (TW)

(72) Inventor: Su-Ling Cheng, New Taipei City (TW)

(21) Appl. No.: 14/705,067

(22) Filed: May 6, 2015

(51) Int. Cl. A61D 9/00; A61F 5/01

(52) U.S. Cl. CPC A61D 9/00; A61F 5/0106

(57) ABSTRACT

An animal protection device, comprising: a main body and a plurality of elastic fixing belts. In the animal protection device, the plurality of support rods are inserted into the corresponding insertion bags of the main body. Through the support of the support rods, the knee joint is kept from easy to slip. In addition, a plurality of fixing belts are provided on both sides of the main body. As such, when the animal protection device is installed on the leg of the animal, the fixing belts are wound around the rear of animal's knee in opposite directions, to connect them onto the main body through the connection portions, so that the animal protection device can be wrapped around the animal's leg tightly, to relieve the pains of the animal when it is walking or kneeling.

### BACKGROUND OF THE INVENTION

### Field of the Invention

The present invention relates to an animal protection device, and in particular to an animal protection device, that is capable of assisting animal having leg injury to walk around or run around easily and smoothly.

### THE PRIOR ARTS

Nowadays, along with the trend of remaining single for life or having fewer children, quite a lot of people tend to raise pets to enrich their lives. Among these pets, dogs are raised by most of the people. In general, dogs are considered as the most loyal friends of mankind, and thus they are treated and taken care of like children by most of the people, so that they take dogs out for a walk every day.

The knee joint disease is the most common disease happens to the dogs, that could cause the dogs to feel uncomfortable while walking, running, or knelling down, as such the dog may refuse to walk. The therapy of knee joint disease can be classified into physical therapy or surgical therapy depending on the severity of knee injury or dislocation.

The pet physical therapy is similar to the rehabilitation of human, such as water therapy, supersonic therapy, rehabilitation ball game exercise, or exercising regularly depending on physical conditions, to improve its physiology capability and its ability to move, and to increase the flexibility of the entire joint muscle tissue.

As to the expense of physical therapy, the expense of water therapy is the highest, while exercising regularly requires the least expense. In order to reduce discomfort, the pets having knee joint dislocation tend to lie down, have no intentions to move. Yet not moving for a long time could produce problems of degenerative arthritis and muscle contraction.

In this respect, in the existing Knee Protection For Pet according to the Prior Art, a protection jig can be sleeved on the leg of a pet, to provide protection and heat retention of the leg. The protection jig includes an elastic piece, that is provided with fine and dense fibers having hooked filaments, and that are used to make the protection jig to attach and fix onto the leg of the pet.

Though this kind of protection jig can provide protection and heat retention of the pet's leg, yet when the pet is walking or running, the vibration thus produced will cause the dislocation of the attaching position of the protection jig, as such reducing its effect of protection. In addition, the protection jig can only provide protection and heat retention, yet it is not able to reduce the discomfort of the pet having knee joint disease when it is walking

Therefore, presently, the design and performance of animal protection device is not quite satisfactory, and it has much room for improvement.

### SUMMARY OF THE INVENTION

In view of the problems and drawbacks of the prior art, the present invention provides an animal protection device, to overcome the shortcomings of the prior art.

A major objective of the present invention is to provide an animal protection device, including a main body and at least a fixing belt. The main body includes: an opening, a first support portion, and a second support portion. The opening is disposed in a center of the main body. The first support portion is located laterally on a side of the opening of the main body, and it can be inserted a support rod. The second support portion is located longitudinally on adjacent side of the opening, and is vertical to the first support portion, such that it can be inserted another insertion rod. Through the support of the first and second support rods, the knee joint is kept from easy to move. In addition, a plurality of fixing belts are located on both sides of the main body, a front end of each said fixing belts is provided with a connection portion, such that when the animal protection device is installed onto the leg of the animal, the fixing belts are wound around the rear of the animal's knee in opposite directions, to connect them onto the main body through the connection portions. As such, the animal protection device can be wrapped around the leg of the animal tightly, to relieve the pains of the animal when it is walking or kneeling.

In addition, the animal protection device of the present invention is provided with an elastic connection belt, with its front end disposed a connection portion, such that the animal protection device can be connected to another animal protection device through the respective connection belts. As such, when the assembled animal protection device is installed onto the leg of the animal, the connection belts are put on the buttock of the animal, to prevent the animal protection device from slipping away when the animal is walking or squatting down to cause it to lose the protection function.

Further scope of the applicability of the present invention will become apparent from the detailed descriptions given hereinafter. However, it should be understood that the detailed descriptions and specific examples, while indicating preferred embodiments of the present invention, are given by way of illustration only, since various changes and modifications within the spirit and scope of the present invention will become apparent to those skilled in the art from this detailed descriptions.

### BRIEF DESCRIPTION OF THE DRAWINGS

The related drawings in connection with the detailed descriptions of the present invention to be made later are described briefly as follows, in which:

FIG. 1 is a schematic diagram of an animal protection device according to an embodiment of the present invention;

FIG. 2 is a schematic diagram of a plurality of animal protection devices assembled according to an embodiment of the present invention;

FIG. 3 is a schematic diagram of an animal protection device bound with fixing belts according to an embodiment of the present invention; and

FIG. 4 is a schematic diagram of an animal protection device in application according to an embodiment of the present invention.

### DETAILED DESCRIPTION OF THE PREFERRED EMBODIMENT

The purpose, construction, features, functions and advantages of the present invention can be appreciated and understood more thoroughly through the following detailed description with reference to the attached drawings.

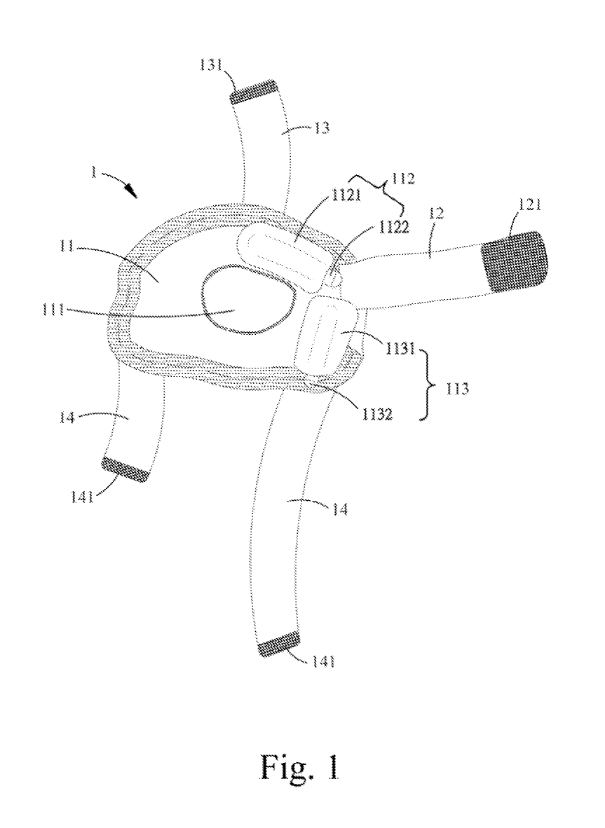

Refer to FIG. 1 for a schematic diagram of an animal protection device according to an embodiment of the present invention. As shown in FIG. 1, the animal protection device 1 mainly includes: a main body 11 and at least a fixing belt 13, 14.

In the descriptions mentioned above, the main body 11 includes: an opening 111, a first support portion 112, and a second support portion 113. Wherein, the first support portion 112 includes an insertion bag 1121, and a support rod 1122; while the second support portion 113 includes an insertion bag 1131, and a support rod 1132. In addition, the animal protection device 1 includes a connection belt 12.

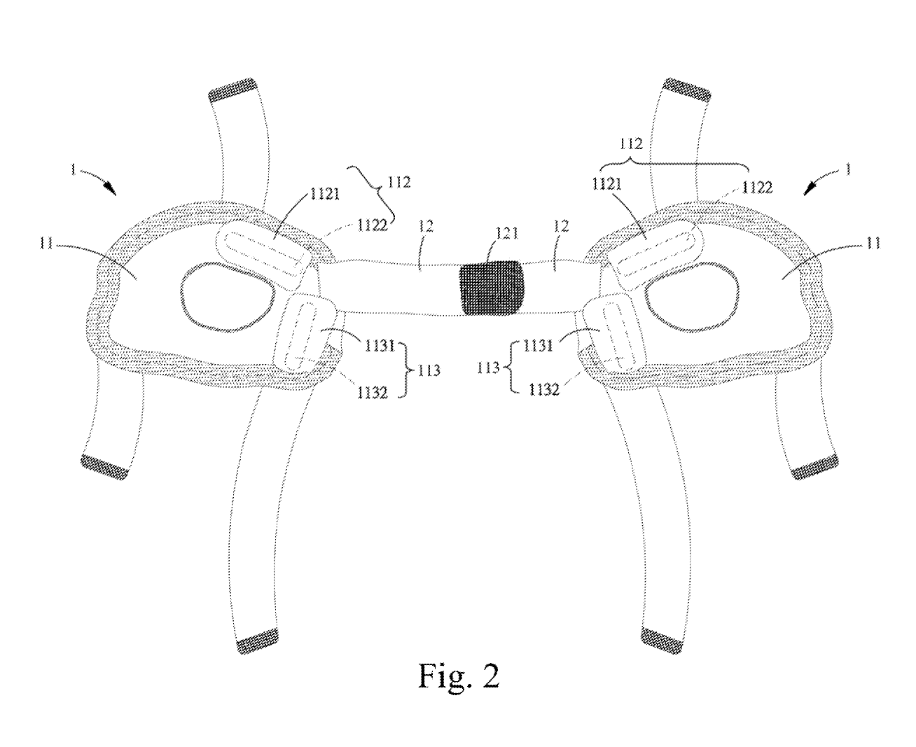

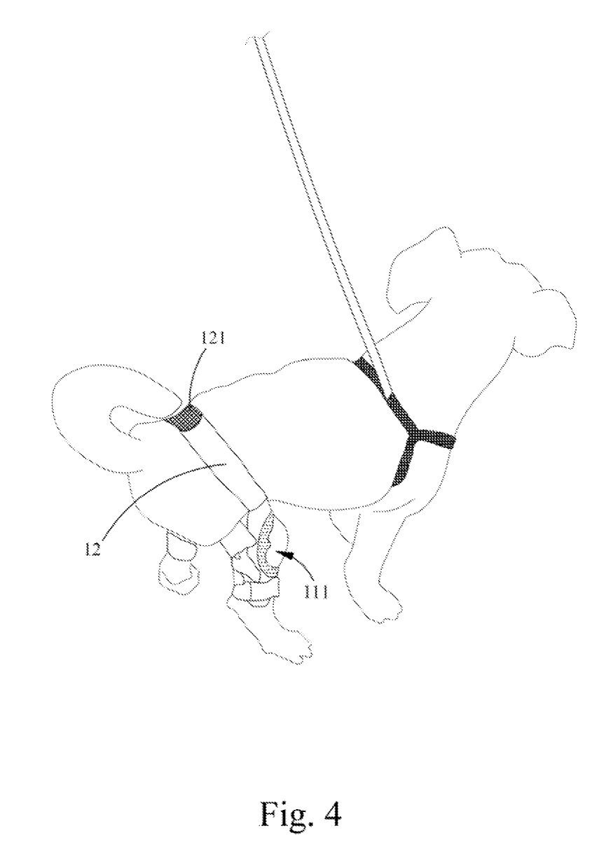

Also, as shown in FIG. 1, the opening 111 is disposed in a center of said main body 11, so that when animal protection device 1 is installed onto a leg of an animal, the opening 111 is pressed against knee bone of the animal. On a side of the main body 11 is provided additionally with an elastic connection belt 12, with its front end provided with a connection portion 121. The animal protection device 1 can be connected to another animal protection device 1 through their respective connection portions 121 (as shown in FIG. 2). As such, when the assembled animal protection device is installed onto the leg of the animal, the connection belts 12 are just put on the buttock of the animal (as shown in FIG. 4), to prevent the animal protection device 1 from slipping away when the animal is walking or squatting down.

Further, as shown in FIG. 1, the first support portion 112 is located laterally on a side of said opening 111 of said main body 11, while the second support portion 113 is located longitudinally on an adjacent side of said opening 11, and is vertical to the first support portion 112. The first support portion 112 and the second support portion 113 are formed respectively by the insertion bags 1121, 1131, and the support rods 1122, 1132, that can be put into the insertion bags 1121, 1131. When the animal protection device 1 is installed onto the animal, the first support portion 112 can be pressed against a ligament located over the knee bone, while the second support portion 113 can be pressed against an inner side of the knee bone. As such, through the support of the first and second support portions 112 and 113, the knee joint is kept from slipping, to relieve the pain of the animal when it is walking or kneeling.

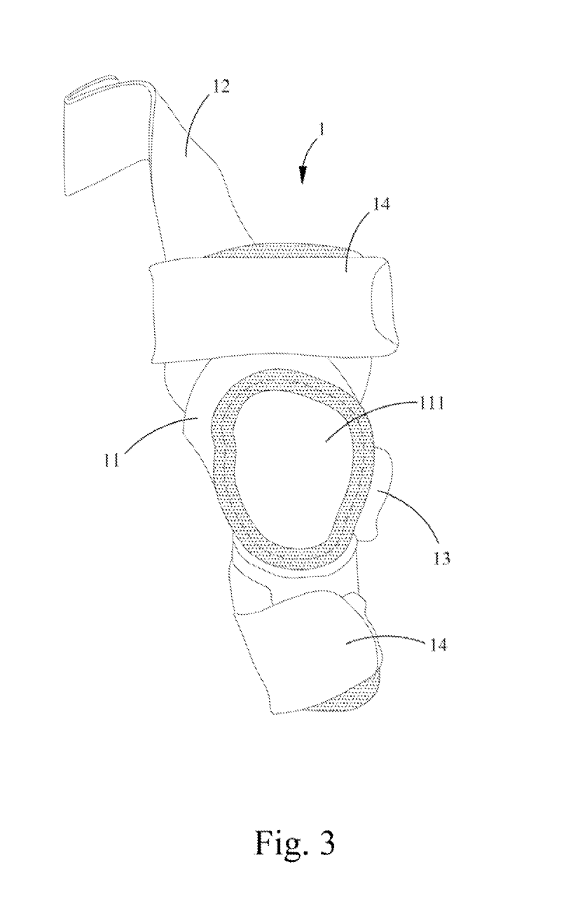

Finally, as shown in FIGS. 1 and 3, an elastic first fixing belt 13 is disposed on a side of the main body 11 vertical to the connection belt 12, while at least an elastic second fixing belt 14 is disposed on a side of the main body 11 opposite to the first fixing belt 13. The fronts of the first fixing belt 13 and the second fixing belt 14 are provided respectively with connection portions 131 and 141, When the animal protection device 1 is installed onto a leg of an animal, the first and second fixing belts 13 and 14 wound around the rear of the animal's knee in opposite directions, to connect them onto the main body 11 through the connection portions 131 and 141, to adjust tight or loose of the first and second fixing belts 13 and 14 on the leg of the animal, so that the animal protection device 1 can be wrapped around the animal's leg tightly (as shown in FIG. 4)

The connection portions 121, 131, and 141 mentioned above can be velcro, a fastening belt, or a snapping button, but the present invention is not limited to this.

The above detailed description of the preferred embodiment is intended to describe more clearly the characteristics and spirit of the present invention. However, the preferred embodiments disclosed above are not intended to be any restrictions to the scope of the present invention. Conversely, its purpose is to include the various changes and equivalent arrangements which are within the scope of the appended claims.

What is claimed is:

1. An animal protection device, comprising:
a main body that includes : an opening, a first support portion, comprising, and a second support portion, said opening is disposed in a center of said main body, so that when said main body is installed onto a leg of an animal, said opening is pressed on a knee bone of said animal, said first support portion is located laterally on a side of said opening of said main body, to press against a ligament located longitudinally over a knee joint of said animal, said second support portion is located on an adjacent side of said opening, and is vertical to said first support portion, to press against an inner side of said knee joint of said animal, and
a plurality of elastic fixing belts, located on both sides of said main body, a front end of each said fixing belt is provided with a connection portion, said fixing belts wound around rear of said knee animal's knee in opposite directions, to connect them onto said main body through said connection portions.

2. The animal protection device as claimed in claim 1, wherein said first support portion and said second support portion are each formed by an insertion bag, and a support rod disposed in said insertion bag.

3. The animal protection device as claimed in claim 1, further comprising: a connection belt is disposed on a side of said main body, and is vertical to said fixing belts, a front end of said connection belt is provided with a connection portion, such that in application, it is connected to said connection belt of other animal protection device.

4. The animal protection device as claimed in claim 1, wherein said connection portion is selected from one of a group consisting of: velcro, a fastening belt, and a snapping button.

5. The animal protection device as claimed in claim 3, wherein said connection portion is selected from one of a group consisting of: velcro, a fastening belt, and a snapping button.
